(19) United States
(12) Patent Application Publication
(10) Pub. No.: US 2024/0058012 A1
(43) Pub. Date: Feb. 22, 2024

(54) MEDICAL DEVICE FOR OCCLUDING A LEFT ATRIAL APPENDAGE

(71) Applicant: BOSTON SCIENTIFIC SCIMED, INC., MAPLE GROVE, MN (US)

(72) Inventors: Joshua Mark Inouye, Brooklyn Park, MN (US); Brian Joseph Tischler, Shoreview, MN (US); Neal Eidenschink, Wayzata, MN (US)

(73) Assignee: BOSTON SCIENTIFIC SCIMED, INC., MAPLE GROVE, MN (US)

(21) Appl. No.: 18/234,023

(22) Filed: Aug. 15, 2023

Related U.S. Application Data
(60) Provisional application No. 63/398,281, filed on Aug. 16, 2022.

(51) Int. Cl. A61B 17/12

(52) U.S. Cl. CPC A61B 17/12122; A61B 17/12168; A61B 2017/00632

(57) ABSTRACT

A medical implant for occluding a left atrial appendage may include an expandable framework configured to shift radially between a first configuration and a second configuration, and an occlusive element secured to the expandable framework. The framework includes a plurality of strut groups, each strut group comprising first and second joints, and first, second, and third struts. The first and second struts move laterally from a first position in the first configuration to a second position in the second configuration. The third strut is configured to shift radially from a first position in the first configuration to a second position in the second configuration. The first and second struts and a majority of the third struts of each strut group may be oriented parallel to each other in the first configuration and nonparallel to each other in the second configuration.

### CROSS REFERENCE TO RELATED APPLICATIONS

This application claims the benefit of priority of U.S. Provisional Application No. 63/398,281 filed Aug. 16, 2022, the entire disclosure of which is hereby incorporated by reference.

### TECHNICAL FIELD

The present disclosure pertains to medical devices and systems, and methods for manufacturing and using medical devices and systems. More particularly, the present disclosure pertains to medical implants for occluding a left atrial appendage.

### BACKGROUND

Diseases and/or medical conditions that impact the cardiovascular system are prevalent throughout the world. Traditionally, treatment of the cardiovascular system was often conducted by directly accessing the impacted part of the system. More recently, less invasive therapies have been developed, and have gained wide acceptance among patients and clinicians.

Atrial fibrillation is a common sustained cardiac arrhythmia affecting over 30 million people worldwide, according to some estimates. Atrial fibrillation is the irregular, chaotic beating of the upper chambers of the heart. Electrical impulses discharge so rapidly that the atrial muscle quivers or fibrillates. Episodes of atrial fibrillation may last a few minutes or several days. The most serious consequence of atrial fibrillation is ischemic stroke. It has been estimated that up to 20% of all strokes are related to atrial fibrillation. Most atrial fibrillation patients, regardless of the severity of their symptoms or frequency of episodes, require treatment to reduce the risk of stroke. The left atrial appendage is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. As a treatment, medical devices have been developed which are positioned in the left atrial appendage and deployed to close off the ostium of the left atrial appendage. Over time, the exposed surface(s) spanning the ostium of the left atrial appendage becomes covered with tissue (a process called endothelization), effectively removing the left atrial appendage from the circulatory system and reducing or eliminating the number of thrombi which may enter the blood stream from the left atrial appendage.

The disclosure relates to medical implants for occluding the left atrial appendage. Of the known medical devices, systems, and methods, each has certain advantages and disadvantages. There is an ongoing need to provide alternative medical devices and systems, as well as alternative methods for manufacturing and using medical devices and systems.

### SUMMARY

In one example, a medical implant for occluding a left atrial appendage may comprise an expandable framework configured to shift radially relative to a central longitudinal axis between a first configuration and a second configuration, and an occlusive element secured to the expandable framework. The expandable framework may include a plurality of strut groups, each strut group comprising a longitudinal axis extending from a first joint to a second joint, a first strut extending from the first joint to the second joint, a second strut extending from the first joint to the second joint, and a third strut disposed between the first strut and the second strut. The first strut may be disposed in a first position in the first configuration and a second position in the second configuration, the first strut moving laterally in a first direction relative to the longitudinal axis from the first position to the second position. The second strut may be disposed in a first position in the first configuration and a second position in the second configuration, the second strut moving laterally in a second direction relative to the longitudinal axis from the first position to the second position. The third strut may be disposed in a first position in the first configuration and is configured to shift radially relative to the longitudinal axis from the first position in the first configuration to a second position in the second configuration.

In addition or alternatively to any example described herein, the second position of the third strut is disposed radially inward from the longitudinal axis.

In addition or alternatively to any example described herein, in the second configuration the third strut is configured to selectively shift radially outward from the second position to a third position radially outward of the first position.

In addition or alternatively to any example described herein, the third strut is selectively shifted from the second position to the third position using an actuation element.

In addition or alternatively to any example described herein, the actuation element is an expandable member disposed within the expandable framework.

In addition or alternatively to any example described herein, the actuation element is at least one tether disposed within the expandable framework.

In addition or alternatively to any example described herein, the third strut is pulled radially inward by the at least one tether as the expandable framework shifts from the first configuration to the second configuration.

In addition or alternatively to any example described herein, subsequently releasing the at least one tether in the second configuration permits the third strut to shift to the third position.

In addition or alternatively to any example described herein, the second position of the third strut is disposed radially outward from the longitudinal axis.

In addition or alternatively to any example described herein, the third strut of each strut group includes at least one anchor member extending radially outward from the third strut in the second configuration.

In addition or alternatively to any example described herein, the plurality of strut groups defines a body region of the expandable framework.

In addition or alternatively to any example described herein, the plurality of strut groups defines a shoulder region of the expandable framework.

In addition or alternatively to any example described herein, in the first configuration, the first strut, the second strut, and the third strut of each strut group are of equal length.

In addition or alternatively to any example described herein, a medical implant for occluding a left atrial appendage may comprise an expandable framework configured to shift radially relative to a central longitudinal axis between a first configuration and a second configuration, and an occlusive element secured to the expandable framework. The expandable framework may include a plurality of strut groups, each strut group of the plurality of strut groups comprising a longitudinal axis extending from a first joint to a second joint, a first strut extending from the first joint to the second joint, a second strut extending from the first joint to the second joint, and a third strut disposed between the first strut and the second strut. The first strut, the second strut, and a majority of the third strut of each strut group of the plurality of strut groups may be oriented parallel to each other in the first configuration and nonparallel to each other in the second configuration. Each of the plurality of strut groups is attached to another strut group of the plurality of strut groups at one of a plurality of body joints.

In addition or alternatively to any example described herein, the third strut is disposed circumferentially between the first strut and the second strut relative to the central longitudinal axis.

In addition or alternatively to any example described herein, the first strut of one of the plurality of strut groups is attached to the second strut of a first adjacent strut group at a first body joint of the plurality of body joints, and the second strut of the one of the plurality of strut groups is attached to the first strut of a second adjacent strut group at a second body joint of the plurality of body joints, the second adjacent strut group disposed on an opposite side of the one of the plurality of strut groups from the first adjacent strut group.

In addition or alternatively to any example described herein, the first strut is disposed in a first position in the first configuration and a second position in the second configuration, the first strut moving laterally in a first direction relative to the longitudinal axis from the first position to the second position. The second strut is disposed in a first position in the first configuration and a second position in the second configuration, the second strut moving laterally in a second direction relative to the longitudinal axis from the first position to the second position. The third strut is disposed in a first position in the first configuration and is configured to shift radially relative to the longitudinal axis from the first position in the first configuration to a second position in the second configuration.

In addition or alternatively to any example described herein, the plurality of strut groups is formed from a single monolithic piece of material.

In addition or alternatively to any example described herein, a medical device system may comprise a catheter, a core wire movably disposed within a lumen of the catheter, and a medical implant for occluding a left atrial appendage releasably connected to a distal portion of the core wire. The medical implant may include an expandable framework configured to shift radially relative to a central longitudinal axis between a first configuration and a second configuration, and an occlusive element secured to the expandable framework. The expandable framework may include a plurality of strut groups, each strut group comprising a longitudinal axis extending from a first joint to a second joint, a first strut extending from the first joint to the second joint, a second strut extending from the first joint to the second joint, and a third strut disposed between the first strut and the second strut.

In addition or alternatively to any example described herein, the expandable framework is disposed in the first configuration when the medical implant is disposed within the lumen of the catheter and the expandable framework is configured to shift toward the second configuration when the medical implant is disposed outside of the lumen of the catheter.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 are side views of an example medical device system;

FIG. 3 is a perspective view illustrating selected aspects of an expandable framework of the medical implant of FIGS. 1-2;

FIG. 4 illustrates one of a plurality of strut groups of the expandable framework in a first configuration;

FIG. 5 illustrates one of the plurality of strut groups of the expandable framework in a second configuration;

FIGS. 6-7 illustrate alternative configurations of a plurality of anchor members of the expandable framework;

FIGS. 8-9 are partial cross-sectional views illustrating selected aspects of one embodiment of the expandable framework in the first configuration and the second configuration;

FIGS. 10-11 are partial cross-sectional views illustrating selected aspects of one embodiment of the expandable framework in the first configuration and the second configuration;

FIGS. 12-13 are partial cross-sectional views illustrating selected aspects of one embodiment of the expandable framework in the first configuration and the second configuration;

FIG. 14 is a flat pattern view illustrating selected aspects of an exemplary embodiment of the expandable framework in the first configuration;

FIG. 15 illustrates selected aspects of one embodiment of the expandable framework in the second configuration;

FIG. 16 illustrates selected aspects of an alternative configuration of one of a plurality of strut groups of the expandable framework in a first configuration; and

FIG. 17 illustrates selected aspects of the alternative configuration of one of the plurality of strut groups of the expandable framework of FIG. 16 in a second configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate exemplary aspects of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. It shall be understood that the discussion(s) herein may apply equally to any and/or all of the components for which there are more than one, unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean the maximum outer dimension, “radial extent” may be understood to mean the maximum radial dimension, “longitudinal extent” may be understood to mean the maximum longitudinal dimension, etc. Each instance of an “extent” may be different (e.g., axial, longitudinal, lateral, radial, circumferential, etc.) and will be apparent to the skilled person from the context of the individual usage. In some instances, an “extent” may be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of medical implants, systems, and methods of manufacturing the same. It should be noted that in any given figure, some features of the medical implants, systems, and methods may not be shown, or may be shown schematically, for simplicity. Additional details regarding some elements may be illustrated in other figures in greater detail. The devices and/or methods disclosed herein may provide a number of desirable features and benefits as described in more detail below.

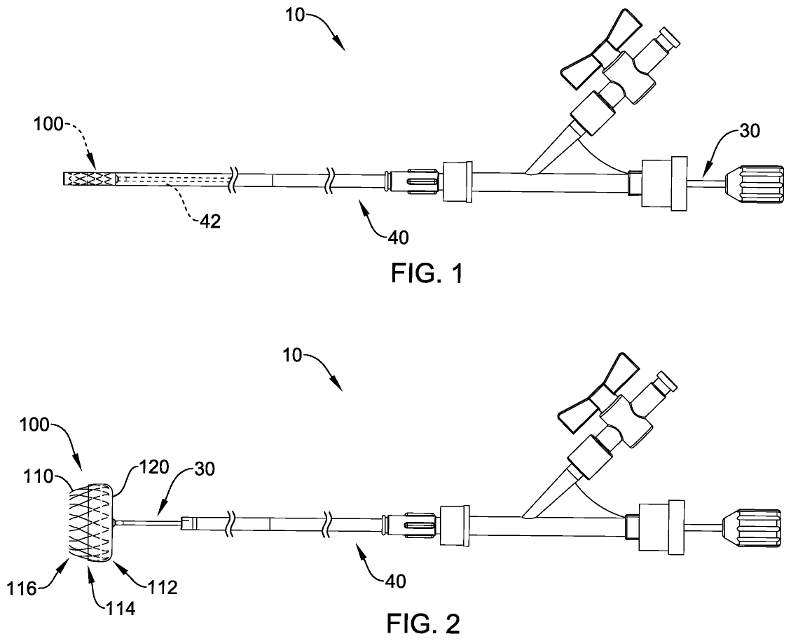

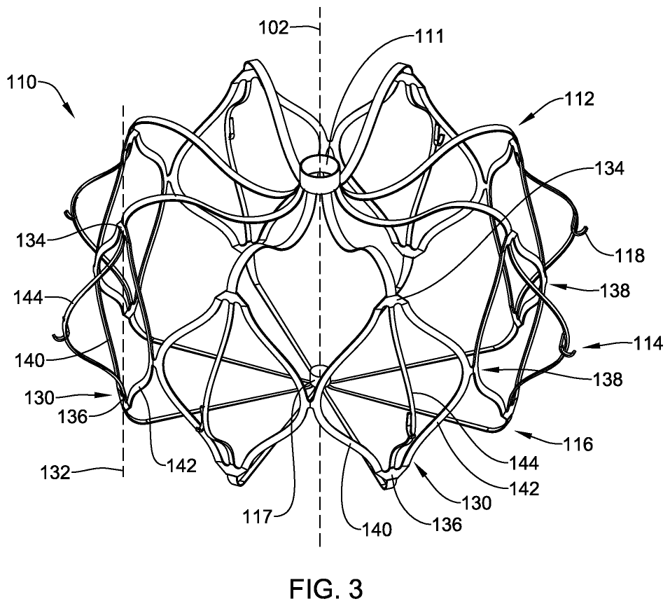

FIGS. 1-2 schematically illustrate selected components and/or arrangements of a medical device system 10. The medical device system 10 may be used to deliver and/or deploy a variety of medical implants (e.g., a cardiovascular medical implant, an occlusive medical implant, etc.) to one or more locations within the anatomy of a patient including but not limited to the heart and/or the vasculature.

The medical device system 10 may include a catheter 40 having a lumen 42 extending from a proximal opening to a distal opening, a core wire 30 movably and/or slidably disposed within the lumen 42, and a medical implant 100 (e.g., a cardiovascular medical implant, an occlusive medical implant, etc.). The medical implant 100 is shown schematically and variations and/or alterations of the example shown are contemplated. The medical implant 100 may be configured to occlude the left atrial appendage of the patient.

The medical implant 100 may include an expandable framework 110 configured to shift between a first configuration (e.g., FIG. 1), wherein the medical implant 100 is disposed within the lumen 42 proximate the distal opening in the first configuration, and a second configuration (e.g., FIG. 2), wherein the medical implant 100 and/or the expandable framework 110 is configured to shift radially relative to a central longitudinal axis 102 (e.g., FIG. 3) between the first configuration and the second configuration when the medical implant 100 is disposed distal of the distal opening of the lumen 42 and/or the catheter 40, and/or when the medical implant 100 is unconstrained by the catheter 40. As shown in FIG. 2, as discussed in additional detail herein, the expandable framework 110 may include a shoulder region 112, a body region 114, and/or a distal region 116.

In some embodiments, the medical implant 100 may include an occlusive element 120 disposed on and/or secured to the expandable framework 110. In some embodiments, the occlusive element 120 may be disposed on, along, and/or over an exterior surface of the expandable framework 110. Alternatively, in some embodiments, the occlusive element 120 may encapsulate and/or embed at least a portion of the expandable framework 110 therein.

In some embodiments, the occlusive element 120 may be and/or may include a porous mesh. In some embodiments, the occlusive element 120 and/or the porous mesh may be a woven structure, a fabric structure, a textile structure, and/or a membrane or film having a plurality of apertures formed therein and/or extending therethrough.

In some embodiments, the occlusive element 120 may cover at least 20% of the expandable framework 110 in the second configuration. In some embodiments, the occlusive element 120 may cover at least 30% of the expandable framework 110 in the second configuration. In some embodiments, the occlusive element 120 may cover at least 40% of the expandable framework 110 in the second configuration. In some embodiments, the occlusive element 120 may cover at least 50% of the expandable framework 110 in the second configuration. In some embodiments, the occlusive element 120 may cover at least 60% of the expandable framework 110 in the second configuration. In some embodiments, the occlusive element 120 may cover at least 70% of the expandable framework 110 in the second configuration. Other configurations are also contemplated.

The medical implant 100 may be disposed at and/or releasably connected to a distal portion of the core wire 30. In some embodiments, the medical implant 100 may be releasably connected to the distal end of the core wire 30. The core wire 30 may be slidably and/or rotatably disposed within the lumen 42 of the catheter 40. In some embodiments, a proximal end of the core wire 30 may extend proximally of a proximal end of the catheter 40 and/or the proximal opening of the lumen 42 for manual manipulation by a clinician or practitioner.

Some suitable, but non-limiting, examples of materials for the medical device system 10, the core wire 30, the catheter 40, and/or the medical implant 100, etc. are discussed below. It is contemplated that any and/or all embodiments and/or configurations of the medical implant 100 disclosed herein may be used in accordance with and/or be associated with the medical device system 10 described above.

FIG. 3 illustrates some selected aspects of the medical implant 100 according to the disclosure. The medical implant 100 includes the expandable framework 110 configured to shift between the first configuration (e.g., FIG. 1) and the second configuration (e.g., FIGS. 2-3). The first configuration may be a radially collapsed configuration and the second configuration may be a radially expanded configuration. The expandable framework 110 may include a proximal hub 111 and/or a plurality of strut groups 130. In some embodiments, the plurality of strut groups 130 may extend from and/or may be connected to the proximal hub 111. In some embodiments, the expandable framework 110 may optionally include a distal hub 117.

The expandable framework 110 may have a central longitudinal axis 102 extending from the proximal hub 111 to the distal hub 117 (where present). The proximal hub 111 of the expandable framework 110 may be configured to releasably attach or connect to the distal end of the core wire 30. The proximal hub 111 of the expandable framework 110 may include a threaded insert (not shown) fixedly attached to the expandable framework 110, the threaded insert being configured to engage a threaded member disposed at the distal end of the core wire of the medical device system 10 to releasably connect the medical implant 100 to the distal end of the core wire 30. The expandable framework 110 may be disposed in the first configuration when the medical implant 100 is disposed within the lumen 42 of the catheter 40 of the medical device system 10. The expandable framework 110 may be configured to shift toward the second configuration when the medical implant 100 is disposed outside of the lumen 42 of the catheter 40 of the medical device system 10 and/or when the medical implant 100 and/or the expandable framework 110 is unconstrained by the catheter 40.

In some embodiments, each strut group of the plurality of strut groups 130 may include a longitudinal axis 132 extending from a first joint 134 to a second joint 136. In some embodiments, each strut group of the plurality of strut groups 130 may include a first strut 140 extending from the first joint 134 to the second joint 136, a second strut 142 extending from the first joint 134 to the second joint 136, and a third strut 144 extending from the first joint 134 to the second joint 136. The third strut 144 may be disposed circumferentially (relative to the central longitudinal axis 102) between the first strut 140 and the second strut 142. Other configurations are also contemplated.

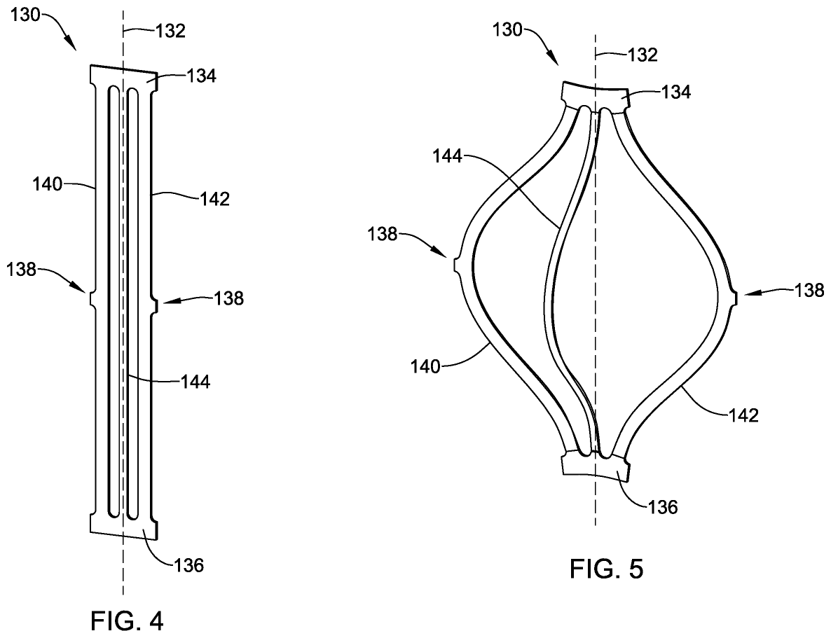

In some embodiments, the first strut 140, the second strut 142, and/or the third strut 144 of each strut group of the plurality of strut groups 130 may be oriented parallel to each other in the first configuration (e.g., FIG. 1, FIG. 4) and nonparallel to each other in the second configuration (e.g., FIG. 2, FIG. 5). In some embodiments, the first strut 140, the second strut 142, and/or the third strut 144 may be generally coplanar in the first configuration. In some embodiments, the first strut 140, the second strut 142, and/or the third strut 144 may be disposed along a curve or an arc around the central longitudinal axis 102 in the first configuration. Each strut group of the plurality of strut groups 130 may be fixedly attached to another strut group of the plurality of strut groups 130 at one of a plurality of body joints 138. Some additional details regarding the plurality of strut groups 130 and individual struts thereof are discussed below.

As may be seen in FIG. 3, in at least some embodiments, the first strut 140 of one of the plurality of strut groups 130 may be fixedly attached to the second strut 142 of a first circumferentially adjacent strut group of the plurality of strut groups 130 at a first body joint of the plurality of body joints 138, and the second strut 142 of the one of the plurality of strut groups 130 may be fixedly attached to the first strut 140 of a second circumferentially adjacent strut group of the plurality of strut groups 130 at a second body joint of the plurality of body joints 138. The second circumferentially adjacent strut group of the plurality of strut groups 130 may be disposed on an opposite side of the one of the plurality of strut groups 130 from the first circumferentially adjacent strut group of the plurality of strut groups 130. In at least some embodiments, the plurality of strut groups 130 may be formed from a single monolithic piece of material (e.g., a tubular member, a flat sheet, etc.). In some embodiments, the expandable framework 110 as a whole may be formed from a single monolithic piece of material (e.g., a tubular member, a flat sheet, etc.). Other configurations are also contemplated.

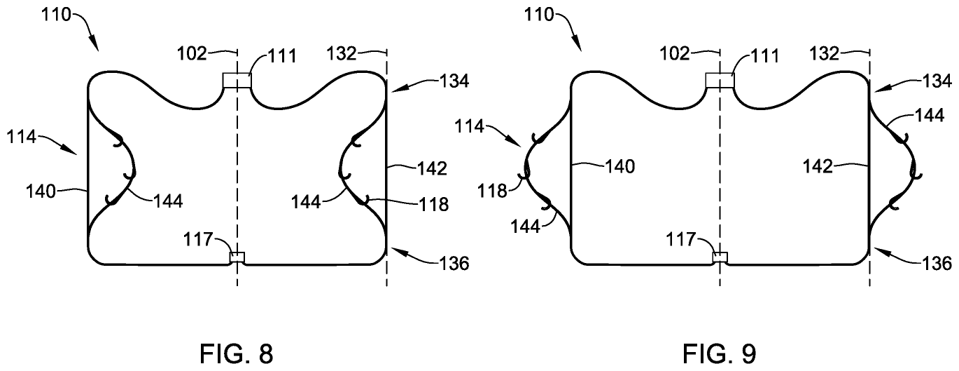

In at least some embodiments, the first joint 134 may be disposed proximate a proximal end and/or the shoulder region 112 of the expandable framework 110. In some embodiments, the first joint 134 may be disposed closer to the proximal end and/or the shoulder region 112 of the expandable framework 110 than the second joint 136. In some embodiments, the second joint 136 may be disposed proximate a distal end and/or the distal region 116 of the expandable framework 110. In some embodiments, the second joint 136 may be disposed closer to the distal end and/or the distal region 116 of the expandable framework 110 than the first joint 134. In some embodiments, the plurality of strut groups 130 may define and/or may at least partially define the body region 114 of the expandable framework 110, as seen in FIG. 3, and additionally shown with respect to FIGS. 8-13, which are discussed in more detail below.

Turning now to FIG. 4, which illustrates a portion of the expandable framework 110 and/or one strut group of the plurality of strut groups 130, the first strut 140 may be disposed in a first position (e.g., FIG. 1, FIG. 4) in the first configuration. The second strut 142 may be disposed in a first position (e.g., FIG. 1, FIG. 4) in the first configuration. The third strut 144 may be disposed in a first position (e.g., FIG. 1, FIG. 4) in the first configuration. In some embodiments, the third strut 144 may be at least partially coaxial and/or coincident with the longitudinal axis 132 in the first position and/or the first configuration. In some embodiments, the third strut 144 may be completely coaxial and/or coincident with the longitudinal axis 132 in the first position and/or the first configuration. As may be seen in FIG. 4, the third strut 144 may be disposed between the first strut 140 and the second strut 142. In some embodiments, the third strut 144 may be disposed circumferentially between the first strut 140 and the second strut 142 with respect to the central longitudinal axis 102 (e.g., FIG. 3). In some embodiments, the first strut 140 and the second strut 142 within the one strut group of the plurality of strut groups 130 may be disposed on opposite sides of the longitudinal axis 132 from each other.

In some embodiments, the first strut 140 may be disposed in a second position in the second configuration. For the purpose of illustration, the point of view of directions, orientations, and/or directional vectors discussed relative to the longitudinal axis 132 shall be understood to be as viewed from outside of the expandable framework 110 looking in toward the central longitudinal axis 102. In some embodiments, the central longitudinal axis 102 and the longitudinal axis 132 may be parallel in the first configuration (e.g., the radially collapsed configuration). In some embodiments, the central longitudinal axis 102 and the longitudinal axis 132 may be parallel in the second configuration (e.g., the radially expanded configuration). In some embodiments, the central longitudinal axis 102 and the longitudinal axis 132 may be nonparallel in the second configuration (e.g., the radially expanded configuration).

In at least some embodiments, the longitudinal axis 132 of one of the plurality of strut groups 130 and the central longitudinal axis 102 may be defined within a common plane containing both axes. As such, the longitudinal axis 132 of each strut group and the central longitudinal axis 102 may be defined within one common plane. Overall, the expandable framework 110 and/or the plurality of strut groups 130 may include and/or define a plurality of common planes equal in number/quantity to the total number of strut groups in the plurality of strut groups 130. Each of the plurality of common planes may include the central longitudinal axis 102 and thus the plurality of common planes may form an array of planes extending outward from the central longitudinal axis 102. In some embodiments, the longitudinal axis 132 of more than one strut group (e.g., two strut groups) may be disposed within one common plane along with the central longitudinal axis 102.

In some embodiments, the first strut 140 may move laterally in a first direction relative to the longitudinal axis 132 from the first position to the second position as viewed from outside of the expandable framework 110 looking in toward the central longitudinal axis 102. In some embodiments, the first strut 140 may move circumferentially in a first direction around and/or relative to the central longitudinal axis 102 (e.g., FIG. 3) from the first position to the second position. In some embodiments, the second strut 142 may be disposed in a second position in the second configuration. In some embodiments, the second strut 142 may move laterally in a second direction relative to the longitudinal axis 132 from the first position to the second position as viewed from outside of the expandable framework 110 looking in toward the central longitudinal axis 102. In some embodiments, the second strut 142 may move circumferentially in a second direction around and/or relative to the central longitudinal axis 102 (e.g., FIG. 3) from the first position to the second position. In at least some embodiments, the second direction may be opposite the first direction relative to the longitudinal axis 132 as viewed from outside of the expandable framework 110 looking in toward the central longitudinal axis 102. In some embodiments, the second direction may be circumferentially opposite the first direction around and/or relative to the central longitudinal axis 102.

In some embodiments, the third strut 144 may be configured to shift radially relative to the longitudinal axis 132 from the first position in the first configuration to a second position in the second configuration as viewed from outside of the expandable framework 110 looking in toward the central longitudinal axis 102. In some embodiments, the third strut 144 may be configured to shift radially relative to the central longitudinal axis 102 from the first position in the first configuration to a second position in the second configuration. In some embodiments, the second position of the third strut 144 may be disposed radially outward from the first position and/or the longitudinal axis 132, as seen in FIG. 5, and as viewed from outside of the expandable framework 110 looking in toward the central longitudinal axis 102. Other configurations are also possible, some of which are discussed herein with respect to FIGS. 8-13.

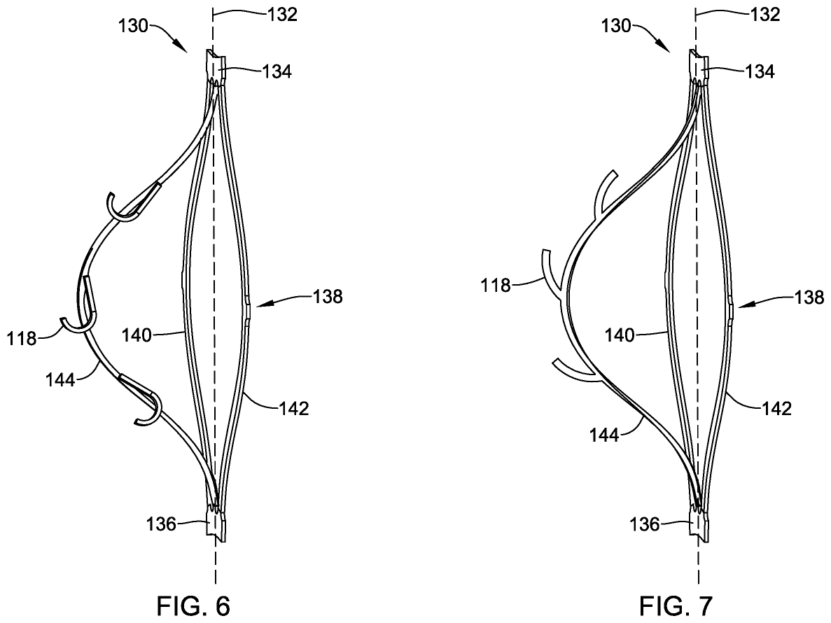

In some embodiments, the expandable framework 110 and/or the third strut 144 may include at least one anchor member 118 each having a free end extending radially outward from the expandable framework 110 and/or the third strut 144 in the second configuration and being connected to the expandable framework 110 and/or the third strut 144 at a base. The at least one anchor member 118 is configured to engage a wall of a left atrial appendage in the second configuration. In some embodiments, one or more of the at least one anchor member 118 extends through the occlusive element 120, as shown in FIG. 2. In some embodiments, the third strut 144 of some strut groups of the plurality of strut groups 130 may include at least one anchor member 118, as seen in FIG. 3. In some embodiments, the third strut 144 of each strut group of the plurality of strut groups 130 may include at least one anchor member 118. In some embodiments, the at least one anchor member 118 may include a plurality of anchor members (e.g., two anchor members, three anchor members, etc.), as seen in FIGS. 6-7. In one non-limiting example, FIG. 6 shows the at least one anchor member 118 fixedly attached to the third strut 144 and shaped in a J-hook configuration in the second configuration. In another non-limiting example, FIG. 7 shows the at least one anchor member 118 fixedly attached to the third strut 144 and extending proximally from the third strut 144 at an oblique angle to the third strut 144 in the second configuration. In some embodiments, the at least one anchor member 118 may be formed separately from the expandable framework 110 and subsequently fixedly attached thereto. In some embodiments, the at least one anchor member 118 may be integrally formed with the expandable framework 110 and/or the third strut 144 as a single monolithic structure. Alternatively, in some embodiments, the expandable framework 110 and/or the third strut 144 may be devoid of any anchor members extending and/or projecting radially outward in the second configuration.

In some embodiments, the second position of the third strut 144 may be disposed radially inward from the first position and/or the longitudinal axis 132, as shown in FIGS. 8, 10, and 12. In some embodiments, in the second configuration, the third strut 144 may be configured to selectively shift radially outward from the second position (e.g., FIGS. 8, 10, and 12) to a third position radially outward of the first position and/or the longitudinal axis 132, as shown in FIGS. 9, 11, and 13. In some embodiments, the third strut 144 may be bistable such that the third strut 144 may be stable and/or may be self-biased to be in the second position and/or the third position. In the first position and/or the first configuration, tension on the third strut 144 may keep and/or maintain the third strut 144 in the first position. As the expandable framework 110 shifts from the first configuration toward the second configuration, compressive force may be exerted on the third strut 144, thereby destabilizing the third strut 144 and causing it to shift away from the first position and/or the longitudinal axis 132 toward the second position. As discussed herein, in some embodiments, the second position of the third strut 144 may be radially outward of the first position and/or the longitudinal axis 132, and in some embodiments, the second position of the third strut 144 may be radially inward of the first position and/or the longitudinal axis 132. In the examples shown in FIGS. 8-13, the second position of the third strut 144 is radially inward of the first position and/or the longitudinal axis 132, and the third position of the third strut 144 is radially outward of the first position and/or the longitudinal axis 132. In some embodiments, an external force may be required to shift the third strut 144 from the second position to the third position, or vice versa.

In the examples shown in FIGS. 8-13, the at least one anchor member 118 may be disposed within an interior of the expandable framework 110 and/or may be spaced away from tissue at a treatment site (e.g., a wall of the left atrial appendage) when the third strut 144 is in the second position. This may be beneficial when placing the medical implant 100 because the medical implant 100 may be repositioned, if necessary, while minimizing injury and/or damage to the tissue (e.g., the wall of the left atrial appendage) prior to anchoring and release from the core wire 30. Once the practitioner is satisfied with placement with the medical implant 100, which may be verified using a suitable imaging means, the third strut 144 may be selectively shifted to the third position, wherein the at least one anchor member 118 extends radially outward from the expandable framework 110 and/or engages with the tissue (e.g., the wall of the left atrial appendage) to anchor the medical implant 100 in place and the core wire may then be disconnected from the medical implant 100.

In some embodiments, the third strut 144 and/or the plurality of strut groups 130 may be formed from a shape memory material that responds to an external stimulus, such as temperature or an electrical current/voltage for example. Other configurations are also contemplated. In FIGS. 8-9, the third strut 144 may be seen shifting from the second position (e.g., FIG. 8) to the third position (e.g., FIG. 9) in response to the external stimulus. In at least some embodiments, no external structure and/or no additional structure is required to shift the third strut 144 from the second position to the third position.

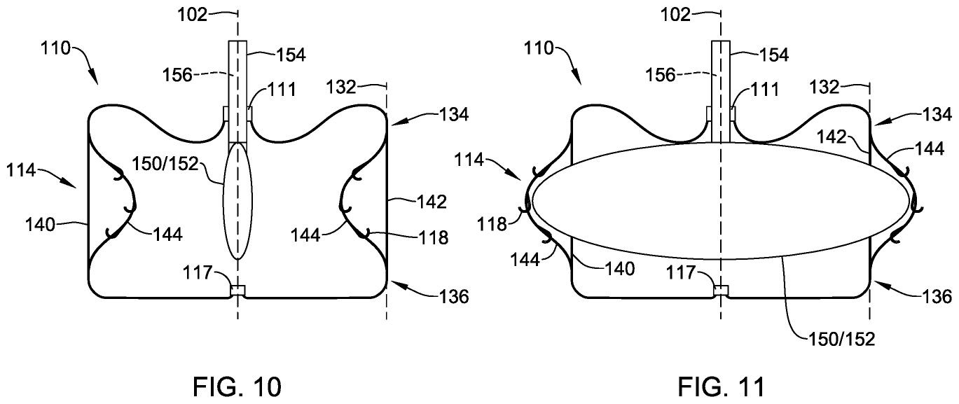

In some embodiments, the third strut 144 may be selectively shifted from the second position to the third position using an actuation element 150. In some embodiments, the actuation element 150 may be an expandable member 152 disposed within the expandable framework 110, as seen in FIGS. 10-11. In some embodiments, the medical implant 100 may include an elongate shaft 154 coupled to the expandable member 152. The elongate shaft 154 may extend within, through, and/or alongside the core wire 30. The elongate shaft 154 may extend through the proximal hub 111 to the expandable member 152. In some embodiments, the expandable member 152 may be mechanically expandable. In some embodiments, the expandable member 152 may be inflatable using a fluid or gas. In at least some embodiments, the expandable member 152 may be an inflatable balloon and the elongate shaft 154 may include an inflation lumen 156 extending therein in fluid communication with the inflatable balloon.

As discussed above, shifting the expandable framework 110 from the first configuration to the second configuration may cause the third strut 144 to shift from the first position to the second position, as shown in FIG. 10. Once placement is confirmed, the expandable member 152 may be expanded from a delivery configuration (e.g., FIG. 10) to an expanded configuration (e.g., FIG. 11), thereby exerting a force against the third strut 144 to selectively shift the third strut 144 from the second position to the third position, as shown in FIG. 11. After anchoring the medical implant and/or after shifting the third strut 144 from the second position to the third position, the expandable member 152 may be collapsed back to the delivery configuration and/or removed from the medical implant 100, if appropriate.

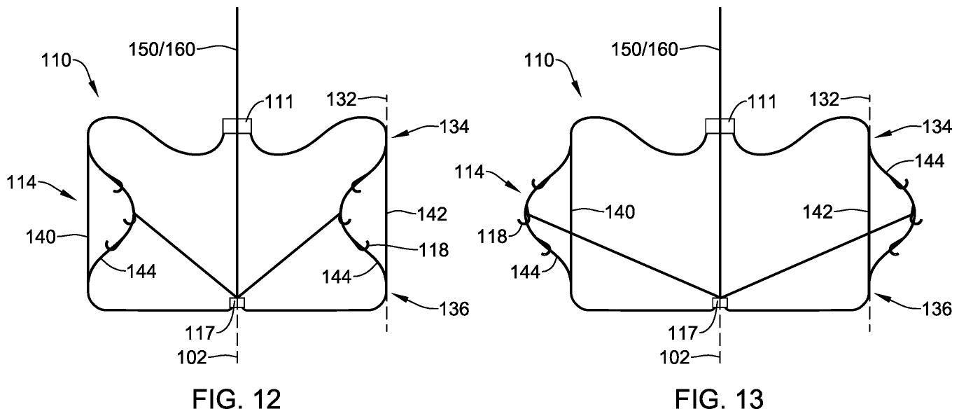

In some embodiments, the actuation element 150 may be at least one tether 160 disposed and/or extending within the expandable framework 110, as seen in FIGS. 12-13. The at least one tether 160 may extend within, through, and/or alongside the core wire 30. The at least one tether 160 may extend through the proximal hub 111 to the distal hub 117 and from the distal hub 117 to the third strut 144. In some embodiments, the distal hub 117 may include an aperture (e.g., a hole or an eyelet) that the at least one tether 160 passes through. In some embodiments, the distal hub 117 may include a pin or a shaft that the at least one tether 160 passes around. Other configurations are also contemplated.

In some embodiments, each strut group of the plurality of strut groups 130 may have one tether of the at least one tether 160 associated therewith. For example, and without limitation, a medical implant 100 having eight strut groups may have at least one tether 160 associated therewith. The quantity eight is chosen merely as an example, and other quantities are also contemplated. In some embodiments, the medical implant 100 having eight strut groups may include eight individual tethers—one coupled to the third strut 144 of each strut group of the plurality of strut groups 130. In some embodiments, the medical implant 100 having eight strut groups (and/or the at least one tether 160 associated therewith) may include one core tether extending to the distal hub 117 and eight distal tethers, wherein one distal tether extends from the one core tether to the third strut 144 of each strut group of the plurality of strut groups 130. Other configurations are also contemplated.

In some embodiments, the third strut 144 may be self-biased toward the third position, and the third strut 144 may be held or retained in the second position by tension applied to the at least one tether 160 during deployment of the medical implant 100 and/or as the expandable framework 110 is shifted from the first configuration to the second configuration. In some embodiments, the third strut 144 may be self-biased toward the third position, and the third strut 144 may be pulled radially inward by the at least one tether 160 toward and/or to the second position as the expandable framework 110 is shifted from the first configuration to the second configuration. As discussed above, shifting the expandable framework 110 from the first configuration to the second configuration may cause the third strut 144 to shift from the first position to the second position, as shown in FIG. 12. Tension on the at least one tether 160 may hold and/or retain the third strut 144 in the second position as the expandable framework 110 is shifted to the second configuration.

Once placement is confirmed, the at least one tether 160 may be released in the second configuration, thereby allowing the third strut 144 to selectively shift from the second position to the third position, as shown in FIG. 13. The at least one tether 160 may be subsequently removed from the medical implant 100 and/or the patient. In some embodiments, releasing the at least one tether 160 may include cutting the at least one tether 160, easing or releasing tension on the at least one tether 160, releasing one end of the at least one tether 160 as the at least one tether 160 is looped through or around the third strut 144 such that the at least one tether 160 may be pulled through the medical implant 100, etc. Other configurations are also contemplated.

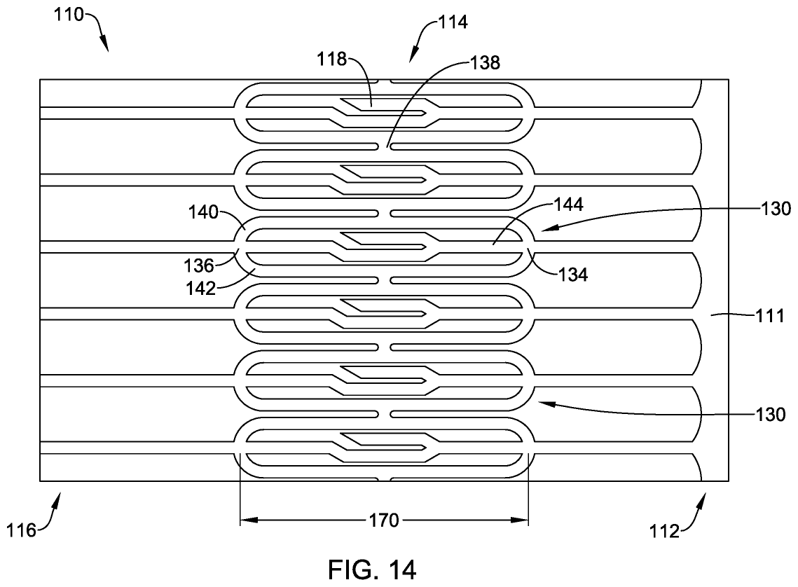

FIG. 14 is a flat pattern view illustrating selected aspects of one example of the expandable framework 110. It shall be understood that FIG. 14 is merely exemplary with respect to some features, and other configurations, including those shown in FIGS. 3-4 are also contemplated, either alone or in combination. In at least some embodiments, in the first configuration, the first strut 140, the second strut 142, and/or the third strut 144 of each strut group of the plurality of strut groups 130 may be of equal length 170, as seen in FIG. 14 and also in FIG. 4. This arrangement and/or configuration may ease and/or improve manufacturability of the plurality of strut groups 130 and/or the expandable framework 110.

In some embodiments, the expandable framework 110 may be formed and/or cut from a unitary tubular member in the first configuration. Alternatively, the expandable framework 110 may be formed and/or cut from a flat sheet of material that is later rolled and/or formed into a tubular member. After forming the flat sheet of material into a tubular member, the tubular member may be welded or otherwise fixedly secured into a tubular shape. In some embodiments, forming and/or cutting the expandable framework 110 may be done via laser, waterjet, machining, etc. Other manufacturing methods and/or processes are also contemplated. It shall be understood that in some embodiments, forming and/or cutting the expandable framework 110 from a unitary tubular member may be preferred and illustration of a flat pattern does not constitute a preference for forming and/or cutting the expandable framework 110 from a flat sheet of material.

In some embodiments, the plurality of strut groups 130 may be integrally formed with a remainder of the expandable framework 110 as a single monolithic structure to form the expandable framework 110 as a whole. In some alternative embodiments, the plurality of strut groups 130 may be formed separately and subsequently fixedly attached the remainder of the expandable framework 110 to form the expandable framework 110 as a whole. In some embodiments, the plurality of strut groups 130 may be fixedly attached to the remainder of the expandable framework 110 by welding, brazing, adhesive bonding, etc. Other configurations are also contemplated. In some embodiments, after forming and/or heat setting the medical implant 100 and/or the expandable framework 110 into the second configuration, the occlusive element 120 may be secured to the expandable framework 110. In some embodiments, at least some of the at least one anchor member 118 may extend through the occlusive element 120 in the second configuration.

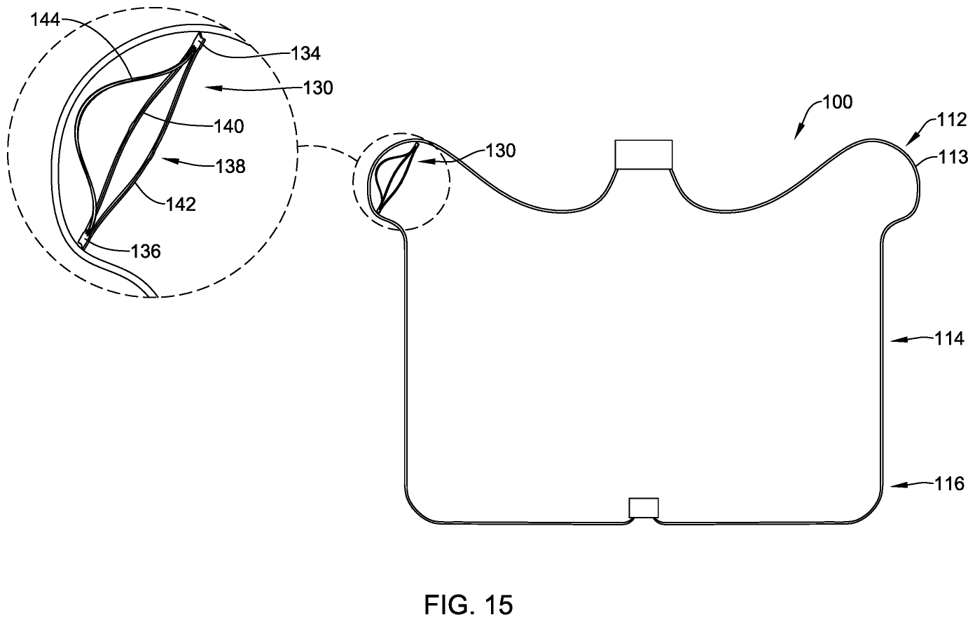

FIG. 15 illustrates an alternative configuration for the expandable framework 110, wherein the plurality of strut groups 130 defines the shoulder region 112 of the expandable framework 110 in the second configuration. In some embodiments, the shoulder region 112 of the expandable framework 110 may define the outermost radial extent of the expandable framework 110 and/or and proximate the proximal end of the medical implant 100 and/or the expandable framework 110 in the second configuration. By positioning and/or applying the plurality of strut groups 130 at the shoulder region 112 of the expandable framework 110 in the second configuration, the medical implant 100 and/or the expandable framework 110 may create a lip 113 extending radially outward of the body region 114. The lip 113 may be positioned proximate and/or within the ostium of the left atrial appendage. In some embodiments, the lip 113 may improve sealing around the perimeter of the medical implant 100 and/or the expandable framework 110 by further engaging and/or applying outward radial force to the wall and/or the ostium of the left atrial appendage. In some embodiments, the third strut 144 of each strut group of the plurality of strut groups 130 may provide some compliance for engagement with the wall and/or the ostium of the left atrial appendage, and/or may improve sealing pressure between the medical implant 100 and the wall and/or the ostium of the left atrial appendage. In some embodiments, the first strut 140 and the second strut 142 of each strut group of the plurality of strut groups 130 may provide and/or maintain higher stiffness at and/or adjacent to the body region 114 of the expandable framework 110 to prevent collapse of the expandable framework 110 and/or to maintain radially outward force against the wall and/or the ostium of the left atrial appendage. Other configurations are also contemplated. For example, in some alternative configurations, the plurality of strut groups 130 may be disposed at and/or along a middle region of the expandable framework 110 and/or a distal region of the expandable framework 110 such that a lip and/or radial protrusion is created around a perimeter of the middle region of the expandable framework 110 and/or the distal region of the expandable framework 110, respectively, in the second configuration to improve sealing between the medical implant 100 and the wall of the left atrial appendage.

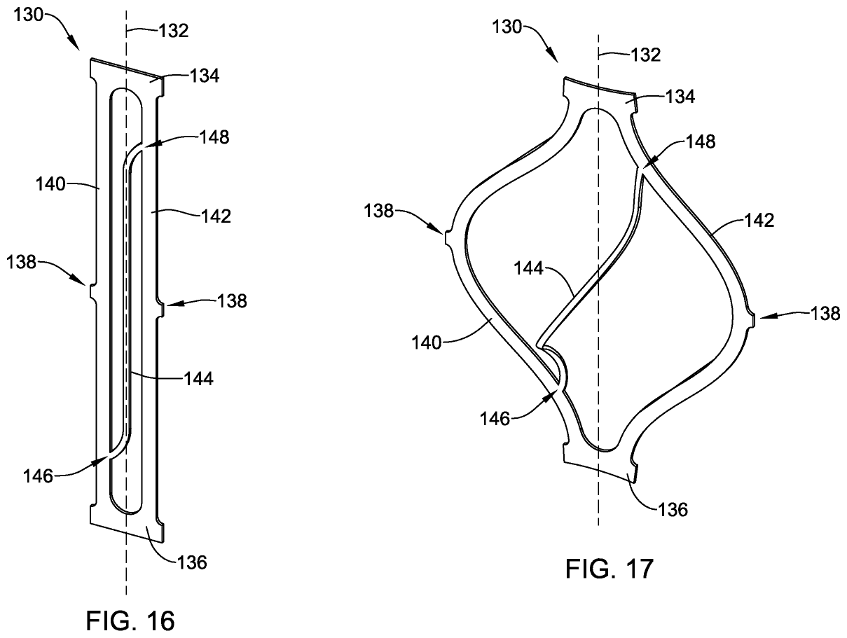

FIGS. 16-17 illustrate an alternative configuration of a portion of the expandable framework 110 and/or one strut group of the plurality of strut groups 130. Similar to other embodiments discussed herein, in some embodiments, each strut group of the plurality of strut groups 130 may include a longitudinal axis 132 extending from a first joint 134 to a second joint 136. In some embodiments, each strut group of the plurality of strut groups 130 may include a first strut 140 extending from the first joint 134 to the second joint 136, a second strut 142 extending from the first joint 134 to the second joint 136, and a third strut 144 disposed circumferentially (relative to the central longitudinal axis 102) between the first strut 140 and the second strut 142.

In some embodiments, the third strut 144 may extend from the first strut 140 to the second strut 142. In some embodiments, the third strut 144 may include a first end 146 fixedly attached to and/or integrally formed with the first strut 140 at a first location disposed between the second joint 136 and a midpoint of the first strut 140. In some embodiments, the third strut 144 may include a second end 148 fixedly attached to and/or integrally formed with the second strut 142 at a second location disposed between the first joint 134 and a midpoint of the second strut 142. In will be appreciated that in some embodiments, the first location and the second location may be reversed such that the first location is disposed between the first joint 134 and the midpoint of the second strut 142 and the second location is disposed between the second joint 136 and the midpoint of the first strut 140. In some embodiments, orientation of the third strut 144 may alternate between adjacent strut groups of the plurality of strut groups 130.

In some embodiments, the first strut 140, the second strut 142, and/or a majority of a length of the third strut 144 of each strut group of the plurality of strut groups 130 may be oriented parallel to each other in the first configuration (e.g., FIG. 1, FIG. 16) and nonparallel to each other in the second configuration (e.g., FIG. 2, FIG. 17). In some embodiments, the first strut 140, the second strut 142, and/or the third strut 144 may be generally coplanar in the first configuration. In some embodiments, the first strut 140, the second strut 142, and/or the third strut 144 may be disposed along a curve or an arc around the central longitudinal axis 102 in the first configuration. In some alternative configurations, the third strut 144 may be oriented nonparallel to the first strut 140 and/or the second strut 142 in the first configuration and in the second configuration. For example, the third strut 144 may be oriented at an oblique angle to the first strut 140 and/or the second strut 142 in the first configuration. Other configurations are also contemplated.

Each strut group of the plurality of strut groups 130 may be fixedly attached to another strut group of the plurality of strut groups 130 at one of a plurality of body joints 138. In some embodiments, the plurality of body joints 138 may be disposed at and/or adjacent to the midpoint(s) of the first strut 140 and/or the second strut 142. Some additional details regarding the plurality of strut groups 130 and individual struts thereof are discussed below.

The first strut 140 may be disposed in a first position (e.g., FIG. 1, FIG. 16) in the first configuration. The second strut 142 may be disposed in a first position (e.g., FIG. 1, FIG. 16) in the first configuration. The third strut 144 may be disposed in a first position (e.g., FIG. 1, FIG. 16) in the first configuration. In some embodiments, the majority of the third strut 144 may be at least partially coaxial and/or coincident with the longitudinal axis 132 in the first position and/or the first configuration. As may be seen in FIG. 16, the third strut 144 may be disposed between and/or may extend between the first strut 140 and the second strut 142. In some embodiments, the third strut 144 may be disposed circumferentially between the first strut 140 and the second strut 142 with respect to the central longitudinal axis 102. In some embodiments, the first strut 140 and the second strut 142 within the one strut group of the plurality of strut groups 130 may be disposed on opposite sides of the longitudinal axis 132 from each other.

In some embodiments, the first strut 140 may be disposed in a second position in the second configuration. In some embodiments, the first strut 140 may move laterally in a first direction relative to the longitudinal axis 132 from the first position to the second position as viewed from outside of the expandable framework 110 looking in toward the central longitudinal axis 102. In some embodiments, the first strut 140 may move circumferentially in a first direction around and/or relative to the central longitudinal axis 102 from the first position to the second position. In some embodiments, the second strut 142 may be disposed in a second position in the second configuration. In some embodiments, the second strut 142 may move laterally in a second direction relative to the longitudinal axis 132 from the first position to the second position as viewed from outside of the expandable framework 110 looking in toward the central longitudinal axis 102. In some embodiments, the second strut 142 may move circumferentially in a second direction around and/or relative to the central longitudinal axis 102 from the first position to the second position. In at least some embodiments, the second direction may be opposite the first direction relative to the longitudinal axis 132 as viewed from outside of the expandable framework 110 looking in toward the central longitudinal axis 102. In some embodiments, the second direction may be circumferentially opposite the first direction around and/or relative to the central longitudinal axis 102.

In some embodiments, the third strut 144 may be configured to shift radially relative to the longitudinal axis 132 from the first position in the first configuration to a second position in the second configuration as viewed from outside of the expandable framework 110 looking in toward the central longitudinal axis 102. In some embodiments, the third strut 144 may be configured to shift radially relative to the central longitudinal axis 102 from the first position in the first configuration to a second position in the second configuration. In some embodiments, the second position of the third strut 144 may be disposed radially outward from the first position and/or the longitudinal axis 132, as seen in FIG. 17, and as viewed from outside of the expandable framework 110 looking in toward the central longitudinal axis 102. Other configurations are also contemplated.

As discussed above, the first end 146 of the third strut 144 may be fixedly attached to and/or integrally formed with the first strut 140 at the first location and the second end 148 of the third strut 144 may be fixedly attached to and/or integrally formed with the second strut 142 at the second location. As the first location is moved closer to or farther from the second joint 136 and/or as the second location is moved closer to or farther from the first joint 134, an orientation of the third strut 144 relative to the longitudinal axis 132, the first strut 140, and/or second strut 142 in the first position and particularly in the second position may change. As the first location is moved closer to the second joint 136 and/or as the second location is moved closer to the first joint 134, the orientation of the third strut 144 may be more vertical relative to the longitudinal axis 132 and/or closer to the orientation illustrated in FIG. 5. As the first location is moved farther from the second joint 136 and/or as the second location is moved farther from the first joint 134, the orientation of the third strut 144 may be more tilted, angled, and/or horizontal relative to the longitudinal axis 132. When the orientation of the third strut 144 becomes more tilted, angled, and/or horizontal relative to the longitudinal axis 132, the third strut 144 and/or the plurality of strut groups 130 may provide improved and/or increased sealing capability when engaged with the wall of the left atrial appendage.

Similar to other embodiments discussed herein, in some embodiments, the expandable framework 110 and/or the third strut 144 may include at least one anchor member each having a free end extending radially outward from the expandable framework 110 and/or the third strut 144 in the second configuration and being connected to the expandable framework 110 and/or the third strut 144 at a base. In some embodiments, the at least one anchor member may be aligned with and/or parallel to the third strut 144. In some embodiments, the at least one anchor member may be twisted and/or bent at the base and/or along its length such that the free end is generally parallel to the longitudinal axis 132 and/or the central longitudinal axis 102. Other configurations, including combinations thereof, are also contemplated. The at least one anchor member is configured to engage a wall of a left atrial appendage in the second configuration. Alternatively, in some embodiments, the expandable framework 110 and/or the third strut 144 may be devoid of any anchor members extending and/or projecting radially outward in the second configuration.

The materials that can be used for the various components of the medical implants and/or medical device systems disclosed herein may include those commonly associated with medical devices. For simplicity purposes, the following discussion refers to the system. However, this is not intended to limit the system, devices, and/or methods described herein, as the discussion may be applied to other elements, members, components, or devices disclosed herein, such as, but not limited to, the expandable framework, the plurality of strut groups, the at least one anchor member, the occlusive element, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system may include a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. In some embodiments, the yarns may be made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible system.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick anti-platelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical implant for occluding a left atrial appendage, comprising:
an expandable framework configured to shift radially relative to a central longitudinal axis between a first configuration and a second configuration; and
an occlusive element secured to the expandable framework;
wherein the expandable framework includes a plurality of strut groups, each strut group comprising a longitudinal axis extending from a first joint to a second joint, a first strut extending from the first joint to the second joint, a second strut extending from the first joint to the second joint, and a third strut disposed between the first strut and the second strut;
wherein the first strut is disposed in a first position in the first configuration and a second position in the second configuration, the first strut moving laterally in a first direction relative to the longitudinal axis from the first position to the second position;
wherein the second strut is disposed in a first position in the first configuration and a second position in the second configuration, the second strut moving laterally in a second direction relative to the longitudinal axis from the first position to the second position;
wherein the third strut is disposed in a first position in the first configuration and is configured to shift radially relative to the longitudinal axis from the first position in the first configuration to a second position in the second configuration.

2. The medical implant of claim 1, wherein the second position of the third strut is disposed radially inward from the longitudinal axis.

3. The medical implant of claim 2, wherein in the second configuration the third strut is configured to selectively shift radially outward from the second position to a third position radially outward of the first position.

4. The medical implant of claim 3, wherein the third strut is selectively shifted from the second position to the third position using an actuation element.

5. The medical implant of claim 4, wherein the actuation element is an expandable member disposed within the expandable framework.

6. The medical implant of claim 4, wherein the actuation element is at least one tether disposed within the expandable framework.

7. The medical implant of claim 6, wherein the third strut is pulled radially inward by the at least one tether as the expandable framework shifts from the first configuration to the second configuration.

8. The medical implant of claim 7, wherein subsequently releasing the at least one tether in the second configuration permits the third strut to shift to the third position.

9. The medical implant of claim 1, wherein the second position of the third strut is disposed radially outward from the longitudinal axis.

10. The medical implant of claim 1, wherein the third strut of each strut group includes at least one anchor member extending radially outward from the third strut in the second configuration.

11. The medical implant of claim 1, wherein the plurality of strut groups defines a body region of the expandable framework.

12. The medical implant of claim 1, wherein the plurality of strut groups defines a shoulder region of the expandable framework.

13. The medical implant of claim 1, wherein in the first configuration, the first strut, the second strut, and the third strut of each strut group are of equal length.

14. A medical implant for occluding a left atrial appendage, comprising:
an expandable framework configured to shift radially relative to a central longitudinal axis between a first configuration and a second configuration; and
an occlusive element secured to the expandable framework;
wherein the expandable framework includes a plurality of strut groups, each strut group of the plurality of strut groups comprising a longitudinal axis extending from a first joint to a second joint, a first strut extending from the first joint to the second joint, a second strut extending from the first joint to the second joint, and a third strut disposed between the first strut and the second strut;
wherein the first strut, the second strut, and a majority of the third strut of each strut group of the plurality of strut groups are oriented parallel to each other in the first configuration and nonparallel to each other in the second configuration;
wherein each of the plurality of strut groups is attached to another strut group of the plurality of strut groups at one of a plurality of body joints.

15. The medical implant of claim 14, wherein the third strut is disposed circumferentially between the first strut and the second strut relative to the central longitudinal axis.

16. The medical implant of claim 14, wherein the first strut of one of the plurality of strut groups is attached to the second strut of a first adjacent strut group at a first body joint of the plurality of body joints, and the second strut of the one of the plurality of strut groups is attached to the first strut of a second adjacent strut group at a second body joint of the plurality of body joints, the second adjacent strut group disposed on an opposite side of the one of the plurality of strut groups from the first adjacent strut group.

17. The medical implant of claim 14, wherein the first strut is disposed in a first position in the first configuration and a second position in the second configuration, the first strut moving laterally in a first direction relative to the longitudinal axis from the first position to the second position;
wherein the second strut is disposed in a first position in the first configuration and a second position in the second configuration, the second strut moving laterally in a second direction relative to the longitudinal axis from the first position to the second position; and
wherein the third strut is disposed in a first position in the first configuration and is configured to shift radially relative to the longitudinal axis from the first position in the first configuration to a second position in the second configuration.

18. The medical implant of claim 14, wherein the plurality of strut groups is formed from a single monolithic piece of material.

19. A medical device system, comprising:
a catheter;
a core wire movably disposed within a lumen of the catheter; and
a medical implant for occluding a left atrial appendage releasably connected to a distal portion of the core wire;
wherein the medical implant includes an expandable framework configured to shift radially relative to a central longitudinal axis between a first configuration and a second configuration, and an occlusive element secured to the expandable framework;
wherein the expandable framework includes a plurality of strut groups, each strut group comprising a longitudinal axis extending from a first joint to a second joint, a first strut extending from the first joint to the second joint, a second strut extending from the first joint to the second joint, and a third strut disposed between the first strut and the second strut.

20. The medical device system of claim 19, wherein the expandable framework is disposed in the first configuration when the medical implant is disposed within the lumen of the catheter and the expandable framework is configured to shift toward the second configuration when the medical implant is disposed outside of the lumen of the catheter.
